(19) United States
(12) Patent Application Publication
(10) Pub. No.: US 2005/0043230 A1
(43) Pub. Date: Feb. 24, 2005

(54) Use of long pentraxin ptx3 for the treatment of fgf-8 mediated tumour diseases

(76) Inventors: Marco Presta, Brescia (IT); Marco Rusnati, Via Valsabbina (IT); Alberto Mantovani, Milan (IT); Barbara Bottazzi, Milan (IT)

Correspondence Address: NIXON & VANDERHYE, PC, 1100 N GLEBE ROAD, 8TH FLOOR, ARLINGTON, VA, 22201-4714, US

(21) Appl. No.: 10/492,130

(22) PCT Filed: Apr. 4, 2003
(86) PCT No.: PCT/IT03/00207

(30) Foreign Application Priority Data
Apr. 8, 2002 (IT) RM2002A000191

(51) Int. Cl. A61K 38/17

(52) U.S. Cl. 514/12

(57) ABSTRACT

The use is described of the long pentraxin PTX3 (PTX3) or one of its functional derivatives as an agent inhibiting the activity of growth factor FGF-8, for the preparation of a medicine for the treatment of tumour diseases associated with abnormal activation of growth factor FGF-8.

The invention described herein relates to the use of the long pentraxin PTX3 (PTX3) or of one of its functional derivatives for the preparation of a medicine for the prevention and treatment of diseases responding to inhibition of the biological activity of growth factor FGF-8.

Abnormal activation of growth factor FGF-8 causes an increase in mortality in patients suffering from tumours.

FGF-8 is associated with increased angiogenesis in patients suffering from tumours, and increases the mortality in said patients.

This compound has confirmed its ability to stimulate the growth of endothelial cells in vitro.

The first compound with antiangiogenic activity was discovered in cartilage by Henry Brem and Judith Folkman in 1975 (J. Exp. Med. 1975 February 1;141(2):427-39). This discovery prompted the authors to postulate the possibility of being able to intervene in the control of disease processes, such as the development of a tumour and its metastatic spread, by means of selective inhibitors of the neovascularisation of the disease.

Angiogenesis in the adult is normally quiescent, yet it constitutes a normal function, for example in the healing of wounds, or in the reconstruction of the endometrium during the female reproductive cycle.

The angiogenic response is stimulated physiologically when the vascular functions are reduced and the tissue perfusion is inadequate.

More generally, it can be said that angiogenesis, in physiological conditions, constitutes a positive feedback in response to inadequate perfusion, or to a reduced supply of oxygen and nutrients, such as, for example, in the case of occlusion of an artery, in situations of tissue mass growth (for example, the neovascularisation accompanying the formation of muscular tissue); and in the case of an increased work load associated with an increased oxygen and nutrient requirement.

### BACKGROUND TO THE INVENTION

Abnormal activation of growth factor FGF-8 causes an increase in mortality in patients suffering from tumours.

FGF-8 is associated with increased angiogenesis in patients suffering from tumours, and increases the mortality in said patients.

This compound has confirmed its ability to stimulate the growth of endothelial cells in vitro.

It is well known that the growth of a primary tumour is facilitated by factors that stimulate the replication of tumour cells and by factors that stimulate good vascularisation of the tumour tissue. An adequate supply of oxygen and nutrient substances facilitates rapid growth of the tumour itself. It has been demonstrated that the extent of neoangiogenesis constitutes a very unfavourable factor in the prognosis of tumours (van Hinsbergh V W, et al.; Ann. Oncol., 10 Suppl., 4:60-3, 1999; Buolamwini J. K; Curr., Opin., Chem., Biol., 3(4):500-9, 1999 August).

It is equally well known in the neoplastic field that a fundamental stage in the biology of tumour cells is their acquisition of the ability to metastasise.

Tumour cells that metastasise have the ability to lose adhesion to the surrounding structures, and to invade the blood and lymph vessels and colonise other distant tissues where they continue to reproduce themselves.

Metastatic spread also constitutes a critical event in the clinical history of the disease, being the main cause of death due to cancer. It is closely associated with the presence, in the tumour site or in the adjacent areas, of vascular tissue.

In fact, the migration of the neoplastic cells through the surrounding structures allows the cells to reach the intratumoral blood vessels, both those existing previously and those formed by neoangiogenesis, and to enter the bloodstream (Ray J. M., Stetler-Stevenson W G; Eur. Respir. J., 7(11):2062-72, 1994; Stetler-Stevenson W G, et al.; FASEB J., 7(15):1434-41, 1993 December). The presence of lymphatic and blood vessels in the vascular district of the tumour allows the neoplastic cells to move in both sets of vessels.

Growth factor FGF-8 is involved in physiological processes such as embryonic organogenesis (Oncogene 1996 Jul. 4; 13(1):47-53) and in disease processes. For example, in Lab. Invest. 2001 June; 81(6):815-26, it is reported that FGF-8 plays an important role in the development of prostate cancer.

In Cancer. Res 1998 May 15;58(10):2053-6, it is reported that FGF-8 plays an important role in the development of prostate and breast cancer.

In Oncogene 1999 January 28;18(4):1053-60, it is reported that FGF-8 is the first member of the FGF family mainly expressed in breast cancer and that it plays a role in the progression of this type of tumour.

In Int. J. Cancer 2000 December; 1;88(5):718-25, it is reported that FGF-8 plays an important role in the development of ovarian cancer.

In Br. J. Cancer 2001 August; 17;85(4):576-83, it is reported that VEGF (Vascular Endothelial Growth Factor) stimulates tumour neoangiogenesis synergistically in the presence FGF (Fibroblast Growth Factor). In this study, it is reported that patients with prostate tumours expressing VEGF and/or FGF-8 have a reduced survival period as compared to patients not expressing FGF-8 and/or VEGF.

A number of results in vitro, reported in the experimental part here below, have confirmed the angiogenic activity of FGF-8.

As regards the treatment of tumour-type diseases, antiangiogenic therapy presents the following advantages as compared to standard traditional chemotherapy (Cancer Research 1998, 58, 1408-16):
1) greater specificity: its target is the neovascularisation process of the tumour;
2) greater bioavailability: its targets are the endothelial cells, which can be easily reached without any of the problems of traditional chemotherapy which acts directly on the tumour cells;
3) less chemoresistance: this is perhaps the most important advantage of this therapy; in fact, since the target cells are endothelial cells, which, unlike tumour cells, are genetically stable, phenomena of resistance to the drug are hardly likely;
4) less metastatic spread: blockade of the neovascularisation limits the proliferation of tumour cells to other parts of the body via the bloodstream;
5) facilitation of apoptosis: blockade of the vascular network in the tumour leads to a reduction in the supply of oxygen and nutrients to the tumour cells, and in these conditions apoptosis is facilitated;
6) reduced systemic toxicity: toxic effects, such as myelosuppression, gastrointestinal effects and temporary hair loss, which are almost always present with traditional chemotherapy, are not observed with antiangiogenic therapy.

The control of neovascularisation is therefore one of the fundamental elements for the control and treatment of diseases associated with abnormal angiogenesis.

The availability of new therapeutic means capable of specifically inhibiting the biological activity of FGF-8 is an objective of primary importance for the prevention and treatment of tumour diseases caused by abnormal activation of this growth factor.

PTX3 is a protein expressed in various types of cells (Bottazzi et al., J., Biol Chem 1997; 272: 32817-32823), particularly in mononuclear phagocytes and endothelial cells, after exposure to the inflammatory cytokines interleukin 1beta (IL-1beta) and tumour necrosis factor alpha (TNF-alpha).

To date the biological function of PTX3 has yet to be understood.

This protein consists of two structural domains, an N-terminal unrelated to any known molecule, and a C-terminal similar to the short pentraxins such as C-reactive protein (CRP). A strong similarity has been found between human PTX3 (hPTX3) and the animal PTX3.

The PTX3 gene is located on mouse chromosome 3, in a region similar to the human region 3q (q24-28), in agreement with the documented location of hPTX3 in the 3q 25 region. In addition, mouse PTX3 (mPTX3) (Introna M. et al., Blood 87 (1996, 1862-1872) is very similar to hPTX3 on the basis of its organisation, location and sequence (Breviario F. et al., J. Biol. Chem. 267:22190, 1992).

In particular, the degree of identity between the sequences is 82% between the human gene and the mouse gene, and as much as 92% if the conservative substitutions are considered.

The high degree of similarity between the sequence of hPTX3 and that of mPTX3 is a sign of the high degree of conservation of pentraxin during evolution (Pepys M. B., Baltz M. L., Adv. Immunol: 34:141, 1983).

For a review of the pentraxins, see H. Gewurz et al., Current Opinion in Immunology, 1995, 7:54-64.

The international patent application WO99/32516, filed in the name of the current applicant, describes the use of the long pentraxin PTX3 for the therapy of infectious, inflammatory or tumoral diseases. The anticancer activity exerted by PTX 3, described in WO99/32516, is mediated by leukocyte recruitment, i.e. on an immunological basis.

WO99/32516 does not describe or suggest the use of PTX3 as a useful agent for the treatment of diseases associated with abnormal activation of growth factor FGF-8.

U.S. Pat. No. 5,767,252 describes a growth factor of neuronal cells belonging to the pentraxin family (see also the literature cited therein). This patent relates to the neurobiology sector.

### SUMMARY OF THE INVENTION

It has now surprisingly been found that long pentraxin PTX3 is capable of binding FGF-8, but not FGF-4, with high affinity and specificity, and of inhibiting the biological activity of said growth factor FGF-8.

The subject of the invention described herein is therefore the use of long pentraxin PTX3 or one of the derivatives of PTX3 or one of its domains, for the preparation of a medicine for the prevention and treatment of diseases caused by abnormal activation of growth factor FGF-8. In the context of these diseases are included tumour diseases and those caused by abnormal angiogenesis. In the context of the diseases caused by abnormal angiogenesis are included tumour diseases and metastatic proliferation.

### DETAILED DESCRIPTION OF THE INVENTION

What is meant by “long pentraxin PTX3” is any long pentraxin PTX3, i.e. regardless of its natural (human or animal) or synthetic origin.

What is meant by derivative is any functional analogue of long pentraxin PTX3 as defined above that carries at least one mutation, conserving the functional ability to selectively inhibit FGF-8.

The preferred form of long pentraxin PTX3 is human PTX3, the sequence of which is described in WO99/32516.

The diseases related to abnormal activation of growth factor FGF-8 include prostate, ovarian and breast tumours.

A further subject of the present invention is the use of full length cDNA of human PTX3 cloned in a suitable plasmid or viral expression vector in gene therapy protocols for the prevention and treatment of diseases caused by abnormal activation of growth factor FGF-8.

The compound according to the present invention lends itself to use for inhibiting the activity of FGF-8 not only when administered as a recombinant protein, but also when expressed endogenously as a result of gene transfer of its cDNA.

Therapeutic protocols for the treatment of tumour-type diseases are known to envisage the use of a combination of several chemotherapeutic agents or of chemotherapeutic agents plus antiangiogenic agents, so as to reduce the side effects, assuming equal therapeutic efficacy. Long pentraxin PTX3, according to the present invention, constitutes an effective therapeutic agent to be used in combination with anticancer drugs for the treatment of such diseases.

The mechanism of action of the most common anticancer agents is completely different from the mechanism of action of the compound according to the invention; the former, in fact, act mainly as cytotoxic compounds on the tumour cells.

The compound according to the present invention acts via a different mechanism of action and thus exerts its own curative effect (coadjuvant effect) which is added to that of the anticancer agents it is used in combination with.

A further subject of the present invention is therefore the combination of long pentraxin PTX3 and one or more compounds with anticancer activity selected from the group consisting of: topoisomerase inhibitors, alkylating agents, antitubulins, antimetabolite agents, intercalating agents or other compounds with anticancer activity used in therapy, for the treatment of such diseases, characterised in that PTX3 is present as a coadjuvant of the anticancer compound.

As regards the aspects relating to industrial applicability, the combination of PTX3 plus an anticancer compound will be in the form of a pharmaceutical composition or kit. The pharmaceutical composition may contain the combination according to the present invention in a single form and/or dosage unit, or in separate dosage forms. In the latter case, the combination according to the present invention may conveniently be formulated in the form of a kit, that is to say in separate containers containing PTX3, possibly in a mixture with at least one pharmaceutically acceptable excipient or vehicle, and the anticancer agent, respectively, the latter, in turn, suitably formulated for the purposes of administration to a subject needing anticancer treatment.

The combination according to the present invention may also be in the form of a manufactured article, e.g. a pharmaceutical composition or kit as described above, containing instruction items for the use of the combination, such as illustration leaflets or other means of providing information for the co-ordinated use of the combination, where what is meant by co-ordinated use of the aforesaid products is, indifferently, either their co-administration, i.e. the substantially simultaneous or sequential administration of PTX3 and at least one anticancer agent, or the administration of a composition containing a combination or mixture of the aforementioned active ingredients, together with any excipients.

The instructions for co-ordinated use relate to the posology and administration regimen in terms of the distribution of the dosages over time and the dosage amounts. The present invention, however, remains adequately described to the extent to which the dosage and posology shall be determined by the primary care physician on the basis of the disease to be treated and the patient's condition.

Examples of pharmaceutical compositions are indicated in WO 99/32516 and in the references cited therein.

Given here below are a number of experimental data further illustrating the invention.

### EXAMPLE 1

Ability of FGF-8 to Inhibit the Binding of ¹²⁵I-FGF-2 to PTX3 Immobilised Onto Plastic

PTX3 was produced using the method described by Bottazzi et al., 1997, J. Biol. Chem. 272:32817-32823.

Human recombinant FGF-2 was produced and labelled with ¹²⁵I using the method described by Isacchi A. et al. in Proc. Natl. Acad. Sci. U.S.A. (1991), 88, 2628-32.

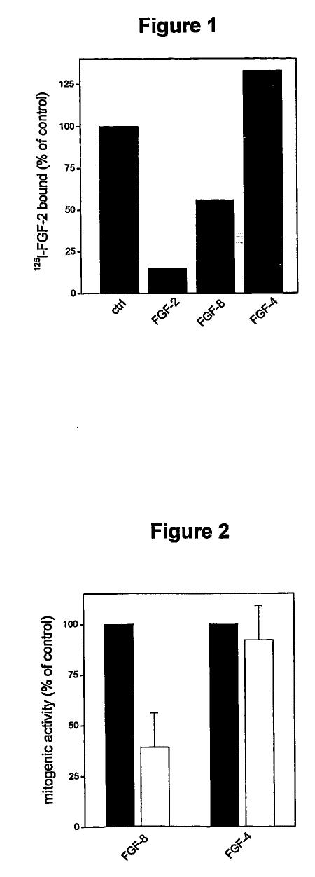

100 μl of NaHCO₃ pH 9.6 containing 1 μg of PTX3 were incubated for 18 hours at 4° C. in 96-well plastic dishes. At the end of the incubation period the wells were washed 3 times with PBS and then incubated for a further 2 hours at ambient temperature with 200 μl of PBS containing 1 mg/ml of BSA. At the end of this second incubation the wells were washed 3 times with PBS. The wells thus prepared were then incubated for 2 hours at ambient temperature with 20 ng/ml of ¹²⁵I-FGF-2 in the absence or presence of 1 μg of unlabelled FGF-2, FGF-4, or FGF-8. At the end of this further incubation, the wells were washed 3 times with PBS. The ¹²⁵I-FGF-2 bound to the wells was recovered by means of two washings with 200 μl of SDS 2% in water and measured by means of a gamma-counter. The results obtained are presented in FIG. 1 and show that unlabelled FGF-8, similarly to FGF-2, inhibits the interaction between ¹²⁵I-FGF-2 and PTX3 immobilised onto plastic. FGF-4, on the other hand, is ineffective.

### EXAMPLE 2

Effect of PTX3 on the Mitogenic Activity of FGF-8 in Endothelial Cells

Transformed bovine foetal aortic endothelial cells GM 7373 were seeded at a concentration of 75,000/cm² in 48-well dishes in MEM Eagle medium containing 10% foetal calf serum (FCS) and were thus incubated for 24 hours at 37° C. At the end of the incubation the cells adhering to the wells were washed twice with MEM-Eagle without FCS and then incubated for a further 24 hours at 37° C. in MEM-Eagle containing 0.4% FCS in the absence or presence of FGF-4 or FGF-8 (both at 30 ng/ml) and PTX3 (1.3 μg/ml). At the end of the incubation the cells were detached with trypsin and counted with a Burker chamber. The results obtained are presented in FIG. 2 and show that PTX3 inhibits the mitogenic and pro-angiogenic activity exerted by FGF-8 in endothelial cells in culture.

In the same figure it will be noted that PTX3 does not inhibit the mitogenic activity of FGF-4.

1. Use of long pentraxin PTX3 or one of its derivatives for the preparation of a medicine for the prevention and treatment of diseases responding to inhibition of the biological activity of growth factor FGF-8.

2. Use according to claim 1, in which said diseases are due to abnormal activation of growth factor FGF-8.

3. Use according to claim 2, in which said abnormal activation of growth factor FGF-8 leads to abnormal angiogenesis.

4. Use according to claim 3, in which the disease caused by abnormal angiogenesis is a tumour.

5. Use according to claim 2, in which said disease is a tumour.

6. Use according to claim 4 or 5, in which the tumour is selected from the group consisting of prostate, ovarian and breast cancer.

7. Use according to claim 3, for the prevention of the occurrence of tumour metastases.

8. Use according to claim 1, in which the long pentraxin PTX3 is naturally occurring PTX3.

9. Use according to claim 8, in which the long pentraxin PTX3 is human PTX3.

10. Use according to claims 1, in which the long pentraxin PTX3 is PTX3 of synthetic origin.

11. Use of cDNA coding for long pentraxin PTX3 or one of its derivatives or one of its domains, to prepare expression vectors containing such cDNA to be used in gene therapy, for the treatment of diseases responding to inhibition of the biological activity of growth factor FGF-8.

12. Use according to claim 11, in which said cDNA containing the gene coding for PTX3 or one of its derivatives is vehicled by a plasmid vector or a viral vector.

13. Use according to claim 11, for the prevention and treatment of tumours.

14. Use according to claim 11, for the prevention and treatment of tumour metastases.

15. Use according to claim 13, in which the tumour is selected from the group consisting of prostate, ovarian and breast cancer.

16. Combination consisting of long pentraxin PTX3 and a compound with anticancer activity.

17. Combination according to claim 16, in which the compound with anticancer activity is selected from the group consisting of topoisomerase inhibitors, alkylating agents, antitubulin agents, antimetabolite agents and intercalating agents.

18. Pharmaceutical composition containing the combination according to claim 16.

19. Use of the combination according to claim 16, for the preparation of a medicine for the treatment of tumour-type diseases, characterised in that the long pentraxin PTX3 is present as a coadjuvant of the compound with anticancer activity.

20. Combination according to claim 16 in the form of a kit.

21. Kit containing a pack of containers consisting of a first container containing a therapeutically efficacious amount of long pentraxin, possibly in a mixture with at least one pharmaceutically acceptable vehicle and/or excipient and of a second container containing a therapeutically efficacious amount of a compound with anticancer activity, possibly in a mixture with at least one pharmaceutically acceptable vehicle and/or excipient.

22. Kit according to claim 21, where said long pentraxin is present in an amount such as to exert a coadjuvant action together with the activity of the anticancer compound.

23. Kit according to claim 21 containing information suitable for the coordinated use of said pentraxin and said compound with anticancer activity.
